(19) United States
(12) Patent Application Publication
(10) Pub. No.: US 2010/0016209 A1
(43) Pub. Date: Jan. 21, 2010

(54) Methods of Reducing Side Effects in Cancer Therapy

(75) Inventors: John Huang, San Carlos, CA (US); Ding Chang, San Carlos, CA (US); Fengping Shan, Shenhe District (CN); Shi-Lung Lo, Attleboro, MA (US)

Correspondence Address: PETERS VERNY , L.L.P., 425 SHERMAN AVENUE, SUITE 230, PALO ALTO, CA, 94306, US

(73) Assignee: PENTA BIOTECH, INC., Union City, CA (US)

(21) Appl. No.: 12/096,152

(22) PCT Filed: Dec. 7, 2006
(86) PCT No.: PCT/US2006/046925

§ 371 Date: Aug. 26, 2009

(30) Foreign Application Priority Data
Dec. 7, 2005 (CN) 200510019964.1

(51) Int. Cl. A61K 38/03; A61P 35/00

(52) U.S. Cl. 514/2

(57) ABSTRACT

The present invention provides a method of decreasing side effects in a human or animal cancer patient due to radiation therapy or chemotherapy. With this method, methionine enkephalin is administered to the patient at least one time per week for a first time period, where the first time period is at least three weeks. In one embodiment, the first time period is one or two months. Methionine enkephalin is then administered to the patient one time per month for a second time period, where the second time period is at least one month, and where the second time period is consecutive to the first time period. In one embodiment, the total term of methionine enkephalin treatment is at least six months. Methionine enkephalin may be administered to the patient at the same time as, before, or after administration of radiation or chemotherapy.

### FIELD OF THE INVENTION

The present invention relates generally to cancer treatment. More particularly, the present invention relates to methods of reducing side effects and increasing therapeutic efficacy of chemo or radiation therapies or combinations of therapies in the treatment of human and animal cancer patients.

### BACKGROUND

Cancers occur in animals and humans due to uncontrollable growth of cells in the body. Cancers cause death in humans in all areas globally. The treatment of a cancer may include surgery, radiation therapy, chemotherapy, biologic response modifiers, or some combination of the above or other therapeutic options. All of these treatment options are directed at killing or eradicating the cancer that exists in the patient's body. Unfortunately, the delivery of the therapy often affects the body's normal organs not involved in cancer. The undesired consequence of affecting an organ not involved with cancer is referred to as a complication of treatment or a side effect.

Side effects of treatment cause inconvenience, discomfort, and occasionally even fatality to patients. Additionally, side effects may prevent doctors from delivering the prescribed dose of therapy at the specific time and schedule of the treatment plan and therefore limit a patient's ability to achieve the best outcome from the treatment by preventing the delivery of therapy at its optimal dose and time.

Common side effects occur with different methods of therapy and also depend on different treatments but usually are: anemia, fatigue, dizziness, paleness, tendency to feel cold, shortness of breath, infections, unexpected bruising, small red spots under the skin, reddish or pinkish urine, black or bloody bowel movement, bleeding from gum or nose, bad headaches, pain in joints and muscles, loss of appetite, a sore mouth, diarrhea or constipation, nausea, vomiting, hair loss, feelings of anxiety, anger or depression and loss of quality of life.

The most serious side effect of chemotherapy is the adverse damage to the immune system of patients undergoing cancer treatment. The immune system protects against infectious agents including viruses, bacteria, fungi, and many parasites. In addition, the immune system is crucial in preventing cancer formation as well as disease states that result form immune impairment, opportunistic infection and autoimmune disorders. Thus, it would be helpful to find a pharmacological agent that would stimulate a suppressed immune system and would prevent the immune system from deterioration during and after cancer therapy. In addition, it would be helpful to find an agent that would reduce side effects due to cancer therapy.

### SUMMARY OF THE INVENTION

The present invention provides a method of using enkephalins to reduce side effects and to enhance therapeutic efficacy in chemotherapy and/or radiation therapy of cancer patients. Taking advantage of their unique multifunctional roles, enkephalins are administered to cancer patients in conjunction with chemotherapy agents and/or radiation before, during, or after each chemotherapy drug or radiation administration. The main ingredient of enkephalins is methionine enkephalin, which was found to be most effective without toxicity.

### DETAILED DESCRIPTION OF THE INVENTION

The present invention provides a method of decreasing side effects in a human or animal cancer patient due to radiation therapy or chemotherapy. With this method, methionine enkephalin is administered to the patient at least one time per week for a first time period, where the first time period is at least three weeks. In one embodiment, the first time period is one or two months. Methionine enkephalin is then administered to the patient one time per month for a second time period, where the second time period is at least one month, and where the second time period is consecutive to the first time period. Preferably, methionine enkephalin is administered to the patient between one time per week and seven times per week during the first time period. In a preferred embodiment, methionine enkephalin is administered to the patient at least two intervals during the first time period. For example, methionine enkephalin may be administered three times per week for the first month, and one time per week for the second month. In one embodiment, the total term of methionine enkephalin treatment is at least six months.

Methionine enkephalin may be administered to the patient at the same time as, before, or after administration of radiation or chemotherapy. In one embodiment, methionine enkephalin is mixed with a chemotherapy agent. For example, methionine enkephalin and the chemotherapy agent may be mixed together and intravenously injected. Methionine enkephalin may be administered by any method, including but not limited to injection (intravenous, subcutaneous, intramuscular, or intradermal), oral droppage, oral spray nasal droppage, nasal spray, tablet, intra-dermal skin pad, eye drops, transdermal pads, or intestine dispersible capsules. Preferably, methionine enkephaline is administered at a dosage of between about 10 μg/kg and about 250 μg/kg bodyweight, more preferably between about 100 μg/kg and 250 μg/kg bodyweight.

Administration of methionine enkephalin reduces many side effects associated with chemotherapy and radiation therapy. Preferably, administration of methionine enkephalin reduces at least one of T lymphocyte depletion, side effects in the gastrointestinal system, side effects in the skin, side effects in the respiratory system, or side effects in the peripheral circulation system.

### 1. Manufacture of Methionine Enkephalin:

Methionine enkephalin (MEK) can be synthesized by either solid phase (on resin) [J. M. Stewart and J. D. Young, Solid Phase Peptide Synthesis, 2^{nd} ed., Pierce Chemical Co., Rockford, Ill. (1984); J. Meienhofer, Hormonal Proteins and Peptides, Vol. 2, Academic Press, New York, (1973)], or solution phase methods [E. Schroder and K. Lubke, The Peptides, Vol. I, Academic Press, New York, (1965)]. The resulting peptides are purified to over 98% using any suitable method known in the art. The peptide can be in the form of acetate, maleate, and other forms as salts. It is preferably lyopholized and stored at −20° C. An injectable solution is prepared by dissolving the lyopholized methionine enkephalin powder in saline at 2.5 mg/ml and sterile-filtering. It can be frozen at −20° C. for longer storage.

### 2. Examples of Clinical Research:

(A) A female patient of 49 years old was a physician in the radiation department. She was diagnosed to have cancer of the “small saliva gland” in 1996. After radiation therapy, the tumor was removed by surgery. In 2001 it was discovered that the tumor had metastasized to her left lung and she went through a second surgery in the lung. In December of 2004, the metastasis extended into the chest, diaphragm and bone and multiple sites in the lung. She was treated with regional radiation and “immuno-therapy”. Starting in the middle of December, she was given MEK intravenously (IV) at a dose of 30 μg/kg bodyweight three times per week for one month. In the next month, she was given one administration of MEK each week at 40 μg/kg. Thereafter, one injection was done for each month at 120 μg/kg for the next 5 months.

During therapy with radiation, chemo, and MEK, her white blood cell counts and lymphocytes decreased to some extent but she had no side effects in the GI, respiratory, skin, and peripheral circulation systems. After the above therapy, her appetite and sleep improved, and her blood pressure, pulse and liver and stomach functions were normal. Her CD3/CD4 sub-group of T lymphocytes (monitored monthly) increased, the total numbers of NK cells increased, and CD3/CD8 and total CD3 subgroups decreased. On CT and MRI examination, the progress of the tumor was found to have slowed and partially disappeared. During the influenza epidemic, she was the only one not infected among all the people around the ward in the hospital. She has been in normal daily life since.

(B) A male patient Mr. Zhong was diagnosed with a kidney cancer and was treated with chemotherapy with a platinum compound and MEK for about two months (July and August 2005). Numbers of his T cell sub-types did not decrease but increased instead, which had not been observed in the history of chemotherapy in the hospital. Mr. Zhong's white blood cells after analyzed by the cell sorter are presented as follows:

| | | Percent Ratio | | Cell Number/μl | | Normal |
|---|---|---|---|---|---|---|
| Test item | | Jul 14 | Aug 12 | Jul 14 | Aug 12 | Value |
| Average CD3 | Total T (CD3+) | 70 | 74 | 1897 | 1368 | 59.4-84.6 |
| Lymphocytes | T + B + NK | 100 | 102 | — | — | 95.0-105.0 |
| Ratio | T Helper Cells/ | 0.45 | 0.48 | — | — | 0.9-3.6 |
| | CD3, CD4/CD3, CD8 | | | | | |
| CD3/CD19 | Total B (CD19+) | 2 | 4 | 54 | 74 | 6.4-22.6 |
| CD3/CD4 | T Helper (CD3+, CD4+) | 21 | 24 | 569 | 447 | 28.5-60.5 |
| CD3/CD8 | T Suppressor (CD3+, CD8+) | 47 | 50 | 1274 | 931 | 11.1-38.3 |
| CD3/CD16+56 | Total NK (CD16+/CD56+) | 28 | 24 | 759 | 447 | 5.6-40.9 |

(C) Therapy in group of patients:
(1) Total patient number: 19
(2) Cancer category: colon cancer—8; ovary cancer—5; lung cancer—4; salivary gland cancer—1; stomach cancer—1. 12 patients were given MEK 3 weeks after chemotherapy; the other 7 patients were not treated with chemotherapy.
(3) Schedule of MEK administration:
First week—10˜30 μg/kg, daily IV (total 7 administrations per week);
Second week—20˜40 μg/kg, ever other day IV (total 3 administrations per week);
Third week—30˜50 μg/kg, once every three days, IV (total 2 administrations per week).
(4) Thereafter, one administration each month, 100˜130 μg/kg for 6 months.
(5) Observation Indicator: analysis of patients' immune cells.
(6) Equipment used: Cytometer—FACS Calibur; Software—Simul Set V3.1.
(7) Results (in average values):

### (a) CD4/CD3(CD8)

### Normal: 0.9-3.6

### Before MEK: 0.61

1 month after MEK: 0
2 months after MEK: 1.62
3 months after MEK: 1.51
4 months after MEK: 1.53
5 months after MEK: 1.80
6 months after MEK: 1.61
(b) Total NK (CD16+CD56) Natural killer cells:

| | |
|---|---|
| In percent of total (normal: 5.6-30.9) | Cells/μl |

| | | |
|---|---|---|
| Before MEK | 16 | 61 |
| 1 month after MEK: | 22 | 177 |
| 2 months after MEK: | 21 | 189 |
| 3 months after MEK: | 29 | 306 |
| 4 months after MEK: | 26 | 349 |
| 5 months after MEK: | 25 | 368 |
| 6 months after MEK: | 27 | 359 |

### (c) Total T Cells

| | |
|---|---|
| In percent of total (normal range: 59.5-84.6) | Cells/μl |

| | | |
|---|---|---|
| Before MEK: | 61 | 367 |
| 1 month after MEK: | 60 | 655 |
| 2 months after MEK: | 66 | 587 |
| 3 months after MEK: | 67 | 539 |
| 4 months after MEK: | 79 | 694 |
| 5 months after MEK: | 73 | 637 |
| 6 months after MEK: | 80 | 568 |

### (d) Total Lymphocytes (T+B+NK)

In percent of total (normal range: 95.0-105.0)

### Before MEK: 87

1 month after MEK: 102
2 months after MEK: 100
3 months after MEK: 104
4 months after MEK: 103
5 months after MEK: 102
6 months after MEK: 103

In the above data, “after MEK” refers to after the end of MEK treatment.

The data in the examples show that in cancer patients treated with MEK as a supplemental agent in conjunction with chemo and/or radiation therapies, their CD4/CD3(CD8), NK (CD16+CD56), and total T cells either were maintained normally or increased after one month of MEK administration. MEK also helped to eliminate or reduce the extent of many side effects among the patients treated. These results prove that the agent MEK, used according to the present invention, plays a critical role in the elimination and/or reduction of side effects as a consequence of radiation and chemo therapies and as a result it improves quality of life of the patient in cancer treatment. It also provides a possibility for further treatment with other drugs or methods in the situation where conventionally the patient is too weak or sick to do so.

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. A method of decreasing side effects in a cancer patient due to radiation therapy or chemotherapy, comprising:
a) administering methionine enkephalin to said patient at least one time per week for a first time period, wherein said first time period is at least three weeks; and
b) administering methionine enkephalin to said patient one time per month for a second time period, wherein said second time period is at least one month, and wherein said second time period is consecutive to said first time period.

2. The method as set forth in claim 1, wherein said methionine enkephalin is administered to said patient between one time per week and seven times per week during said first time period.

3. The method as set forth in claim 1, wherein said methionine enkephalin is administered to said patient at least two time intervals during said first time period.

4. The method as set forth in claim 1, wherein said side effects are at least one of T lymphocyte depletion, side effects in the gastrointestinal system, side effects in the skin, side effects in the respiratory system, or side effects in the peripheral circulation system.

5. The method as set forth in claim 1, wherein said methionine enkephalin is administered to said patient for a total of at least six months.

6. The method as set forth in claim 1, wherein said methionine enkephalin is administered by injection, wherein said injection is intravenous, intramuscular, intradermal, or subcutaneous, oral droppage, oral spray, nasal droppage, nasal spray, tablet, intra-dermal skin pad, transdermal skin pad, eye drops or intestine dispersible capsules.

7. The method as set forth in claim 1, wherein said methionine enkephalin is mixed with a chemotherapy agent.

8. The method as set forth in claim 1, wherein said methionine enkephalin is administered at a dosage of between 10 μg/kg bodyweight and 250 μg/kg bodyweight.

9. The method as set forth in claim 1, wherein said first time period is one or two months.

10. The method as set forth in claim 1, wherein said methionine enkephalin is administered to said patient at least two dosages during said first time period.
